(19) United States
(12) Patent Application Publication
(10) Pub. No.: US 2024/0001145 A1
(43) Pub. Date: Jan. 4, 2024

(54) SYSTEMS AND METHODS FOR RADIATION DOSE PLANNING FOR CARDIAC RADIATION THERAPY

(71) Applicant: Varian Medical Systems, Inc., Palo Alto, CA (US)

(72) Inventors: Tiziano Passerini, Plainsboro, NJ (US); Puneet Sharma, Princeton Junction, NJ (US)

(73) Assignee: Varian Medical Systems, Inc., Palo Alto, CA (US)

(21) Appl. No.: 17/855,257

(22) Filed: Jun. 30, 2022

(51) Int. Cl. A61N 5/10

(52) U.S. Cl. CPC A61N 5/1031; A61N 2005/1035

(57) ABSTRACT

Systems and methods for simulating radiation effect for cardiac ablation can include a processor generating a heart simulation model configured to simulate electrical activities of the heart of the patient. The processor can determine a simulated post-radiation state of the heart of the patient by adjusting the heart simulation model to account for an effect of a radiation treatment plan on simulated electrical activities of the heart of the patient. The processor can simulate, using the adjusted heart simulation model, the simulated post-radiation state of the heart with a stimulation to induce a heart rhythm disorder, determine whether the heart rhythm disorder is induced based on electrical activities generated when simulating the simulated post-radiation state of the heart with the stimulation, and output an indication of whether the heart rhythm disorder is induced. The processor can suggest modifications to the radiation plan if the heart rhythm disorder is induced.

### TECHNICAL FIELD

The present application relates generally to systems and methods for planning of cardiac radiation therapy. Specifically, the present application relates to systems and methods for simulating the effect of radiation on the electrical or electrophysiological properties of the heart, and optimizing or adjusting the radiation dose distribution based on the simulation results to mitigate or eliminate the recurrence of one or more heart pathological conditions. The simulations involve simulating the one or more pathological conditions and checking for potential recurrence.

### BACKGROUND

Cardiac ablation is usually an invasive medical procedure used to treat a variety of heart conditions, such as atrial fibrillation (AFib), atrial flutter, atrial tachycardia, ventricular tachycardia (VT), atrioventricular nodal reentrant tachycardia (AVNRT), paroxysmal supraventricular tachycardia (PSVT), Wolff-Parkinson-White syndrome or heart tumors. The standard radio cardiac procedure involves a doctor inserting a catheter into a patient's body to access the patient's heart. Heat or extreme cold is then applied to destroy abnormal areas of the heart and disrupt the abnormal electrical signals traveling through the heart. The procedure may be risky at least for some types of patients, and typically requires monitoring patients in the intensive care unit afterward. Some of the risks associated with standard radiofrequency ablation include bleeding or infection at the site where the catheter was inserted, blood vessel damage, heart valve damage, new or worsening arrhythmia, slow heart rate, blood clots, stroke or heart attack, pulmonary vein stenosis, damage to the kidneys from contrast used during the procedure and/or death in rare cases.

Cardiac radiotherapy ablation (also referred to as cardiac radioablation) is a noninvasive form of cardiac ablation. Instead of a catheter, a radiation dose is used to target the abnormal areas of the heart to destroy or modify them. The use of radiation relieves some of the risks associated with the invasive procedure and provides relief for high-risk heart patients who most likely have run out of other options. However, cardiac radioablation has its own risks and comes with its own challenges. Among them is the risk of destroying surrounding healthy tissue, particularly in the heart region. Such risk calls for accurate radiation in terms of radiation area(s) and radiation dose(s).

Prior to the radiotherapy ablation procedure, radiotherapy treatment planning is performed. The objective of treatment planning is to optimize radiation angles and/or radiation doses for various angles to ensure that a high radiation dose is delivered to the target region and a low radiation dose is applied to intervening tissues.

### SUMMARY

Embodiments described herein relate to optimizing or improving radiation dose distribution for radiation cardiac ablation in a way to mitigate or eliminate recurrence of a heart rhythm disorder associated with a heart pathological condition. A computer system can generate a patient-specific heart simulation model that is capable of mimicking or reproducing electrical activities of the patient's heart. In the heart simulation model, the electrical activities or activation signals can be modeled using a set of parameters. The computer system can determine the effect of a radiation distribution (or radiation treatment plan) on the set of parameters modeling the electrical activities or signals generated by the heart simulation model, and adjust the set of parameters accordingly. The adjusted heart simulation model represents or corresponds to a post-radiation state of the heart with respect to reproducing electrical activities of the patient's heart. The computer system can virtually stimulate the heart rhythm disorder associated with the heart pathological condition in the adjusted heart simulation model, and check whether the stimulations causes a recurrence of the heart rhythm disorder. If a recurrence of the heart rhythm disorder is detected, the computer system can adjust the radiation distribution (or the radiation treatment plan), e.g., by changing radiation dose values and/or changing a target region. The computer system can iteratively adjust the radiation distribution and the parameters of the heart simulation model, and check for recurrence of the heart rhythm disorder until no recurrence of the heart rhythm disorder is achieved or detected. When no recurrence of the pathological condition is achieved or detected, the corresponding radiation distribution can be used for further planning of the radiation treatment plan.

According to one aspect, a method of simulating radiation effect for cardiac ablation can include one or more processors generating, based on medical images and electrophysiology data of a patient, a heart simulation model of a heart of the patient. The heart simulation model can be configured to simulate electrical activities of the heart of the patient. The method can include the one or more processors determining a simulated post-radiation state of the heart of the patient by adjusting the heart simulation model to account for an effect of a radiation treatment plan on simulated electrical activities of the heart of the patient. The radiation treatment plan specifies radiation doses to be applied to different regions of the heart of the patient. The method can include the one or more processors simulating, using the adjusted heart simulation model, the simulated post-radiation state of the heart with a stimulation to induce a heart rhythm disorder, determining whether the heart rhythm disorder is induced based on electrical activities generated when simulating the simulated post-radiation state of the heart with the stimulation, and outputting an indication of whether the heart rhythm disorder is induced in the simulated post-radiation state of the heart.

In some implementations, the heart rhythm disorder is associated with at least one of ventricular tachycardia (VT), atrial tachycardia, ventricular fibrillation or atrial fibrillation. In some implementations, the electrical activities are defined in the heart simulation model using a plurality of parameters of a plurality of electrical activation signals. Each electrical activation signal can be associated with a corresponding region of the heart of the patient. Adjusting the heart simulation model can include adjusting parameters of electrical activation signals associated with a region of the heart of the patient expected to receive radiation under the radiation treatment plan. Each electrical activation signal can have multiple phases and each phase can be defined by one or more corresponding parameters. Adjusting parameters of the electrical activation signal can include determining changes in parameters of each phase of the electrical activation signal due to an effect of the corresponding radiation dose on the region of the heart of the patient expected to receive radiation, and modifying the parameters of each phase of the electrical activation signal according to the determined changes.

In some implementations, the electrical activities are defined in the heart simulation model using one or more parameters of a conduction velocity. Adjusting the heart simulation model can include adjusting at least one parameter of the one or more parameters of the conduction velocity. In some implementations, determining the simulated post-radiation state of the heart can include identifying tissue of the heart of the patient that is expected to become electrically inert in response to the radiation treatment plan.

In some implementations, simulating the simulated post-radiation state of the heart with the stimulation to induce the heart rhythm disorder can include simulating a virtual catheter-based stimulation in the post-radiation state of the heart.

In some implementations, the method can further include proposing one or more modifications to the radiation treatment plan upon determining that the heart rhythm disorder is induced. The one or more modifications to the radiation treatment plan can include at least one of modifying a radiation dose for a first region of the heart specified in the radiation treatment plan, or modifying a second region of the heart expected to receive radiation under the radiation treatment plan.

According to one other aspect, a system for simulating radiation effect for cardiac ablation can include one or more processors and a memory to store computer code instructions. The computer code instructions when executed cause the one or more processors to generate, based on medical images and electrophysiology data of a patient, a heart simulation model of a heart of the patient. The heart simulation model can be configured to simulate electrical activities of the heart of the patient. The one or more processors can determine a simulated post-radiation state of the heart by adjusting the heart simulation model to account for an effect of a radiation treatment plan on simulated electrical activities of the heart of the patient. The radiation treatment plan specifies radiation doses to be applied to different regions of the heart of the patient. The one or more processors can simulate, using the adjusted heart simulation model, the simulated post-radiation state of the heart with a stimulation to induce a heart rhythm disorder, determine whether the heart rhythm disorder is induced in the simulated post-radiation state of the heart of the patient, and output an indication of whether the heart rhythm disorder is induced in the simulated post-radiation state of the heart of the patient.

In some implementations, the heart rhythm disorder can be associated with at least one of ventricular tachycardia (VT), atrial tachycardia, ventricular fibrillation or atrial fibrillation. In some implementations, the electrical activities can be defined in the heart simulation model using a plurality of parameters of a plurality of electrical activation signals. Each electrical activation signal can be associated with a corresponding region of the heart of the patient. In adjusting the heart simulation model, the one or more processors can adjust parameters of electrical activation signals associated with a region of the heart of the patient expected to receive radiation under the radiation treatment plan. Each electrical activation signal can have multiple phases and each phase can be defined by one or more corresponding parameters. In adjusting parameters of the electrical activation signal, the one or more processors can determine changes in parameters of each phase of the electrical activation signal due to an effect of the corresponding radiation dose on the region of the heart of the patient expected to receive radiation, and modify the parameters of each phase of the electrical activation signal according to the determined changes.

In some implementations, the electrical activities can be defined in the heart simulation model using one or more parameters of a conduction velocity. In adjusting the heart simulation model, the one or more processors can adjust at least one parameter of the one or more parameters of the conduction velocity. In some implementations, in determining the simulated post-radiation state of the heart, the one or more processors can identify tissue of the heart of the patient that is expected to become electrically inert in response to the radiation treatment plan.

In some implementations, in simulating the simulated post-radiation state of the heart with the stimulation to induce the heart rhythm disorder, the one or more processors can simulate a virtual catheter-based stimulation in the post-radiation state of the heart.

In some implementations, the one or more processors can further propose one or more modifications to the radiation treatment plan upon determining that the heart rhythm disorder is induced. The one or more modifications to the radiation treatment plan can include at least one of modifying a radiation dose for a first region of the heart specified in the radiation treatment plan, or modifying a second region of the heart expected to receive radiation under the radiation treatment plan.

According to yet one other aspect, a computer readable medium can include computer code instructions stored thereon. The computer code instructions when executed can cause one or more processors to generate, based on medical images and electrophysiology data of a patient, a heart simulation model of a heart of the patient. The heart simulation model can be configured to simulate electrical activities of the heart of the patient. The one or more processors can determine a simulated post-radiation state of the heart by adjusting the heart simulation model to account for an effect of a radiation treatment plan on simulated electrical activities of the heart of the patient. The radiation treatment plan specifies radiation doses to be applied to different regions of the heart of the patient. The one or more processors can simulate, using the adjusted heart simulation model, the simulated post-radiation state of the heart with a stimulation to induce a heart rhythm disorder, determine whether the heart rhythm disorder is induced in the simulated post-radiation state of the heart of the patient, and output an indication of whether the heart rhythm disorder is induced in the simulated post-radiation state of the heart of the patient.

### BRIEF DESCRIPTION OF THE DRAWINGS

FIG. 1 shows a block diagram illustrating a computer environment for implementing methods and processes described herein, according to an embodiment.

FIG. 2 is a block diagram depicting one implementation of a system architecture, according to an embodiment.

FIG. 3 is a block diagram of a system for simulating radiation effect on electrical properties of a heart, according to an embodiment.

FIG. 4 is a flowchart illustrating a method of simulating effect of radiation on electrical or electrophysiological properties of a heart, according to an embodiment.

Some or all of the figures are schematic representations for purposes of illustration. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### DETAILED DESCRIPTION

Following below are more detailed descriptions of various concepts related to, and implementations of, methods, apparatuses, and systems for predicting locations of heart regions for planning cardiac radiotherapy ablation. The various concepts introduced above and discussed in greater detail below may be implemented in any of numerous ways as the described concepts are not limited to any particular manner of implementation. Examples of specific implementations and applications are provided primarily for illustrative purposes.

Cardiac radiotherapy ablation holds the promise of being a safer and more effective ablation strategy compared to catheter-based ablation, mainly due to its non-invasiveness nature and the potential ability of targeting the full thickness of the myocardium. However, to make such a promise a reality various challenges are still to be overcome. These challenges are mainly due to the strict requirements with regard to radiation accuracy. The heart is composed of multiple structures at risk, including coronary arteries and valves. These structures are to be spared from radiation (or to be subjected to a relatively low radiation) to avoid permanent and serious damage to the heart. At the same time, the treatment aims to apply a sufficiently high radiation dose to abnormal regions of the heart in order to destroy or at least change the characteristics (e.g., electrophysiology properties) of these regions. This tradeoff makes the radiation therapy an intricate and complex task in general, and more so for the heart.

One of the technical challenges associated with cardiac radiation therapy is planning and assessing the efficacy of a radiation plan or a corresponding radiation dose distribution. In other words, given a radiation dose distribution, how can we assess its effect on the patient's heart and determine whether it is sufficient to heal the patient's heart. To assess radiation efficacy, doctors usually attempt to induce VT in the patient's heart using catheter stimuli after the radiation treatment is done. However, such approach is invasive and cannot be used for radiation dose planning since the stimulation is done after the radiation treatment.

In the current disclosure, systems and methods for simulating the effect of radiation on a patient's heart are described. A heart simulation model that is configured to reproduce the heart's electrical activities is used. The effect of radiation is modeled as a variation or adjustment of the parameters of the heart simulation model. Digital catheter stimuli can be applied to the adjusted heart simulation model to determine whether heart rhythm disorder can be induced. If heart rhythm disorder is successfully induced, the radiation dose distribution can be adjusted for better efficacy.

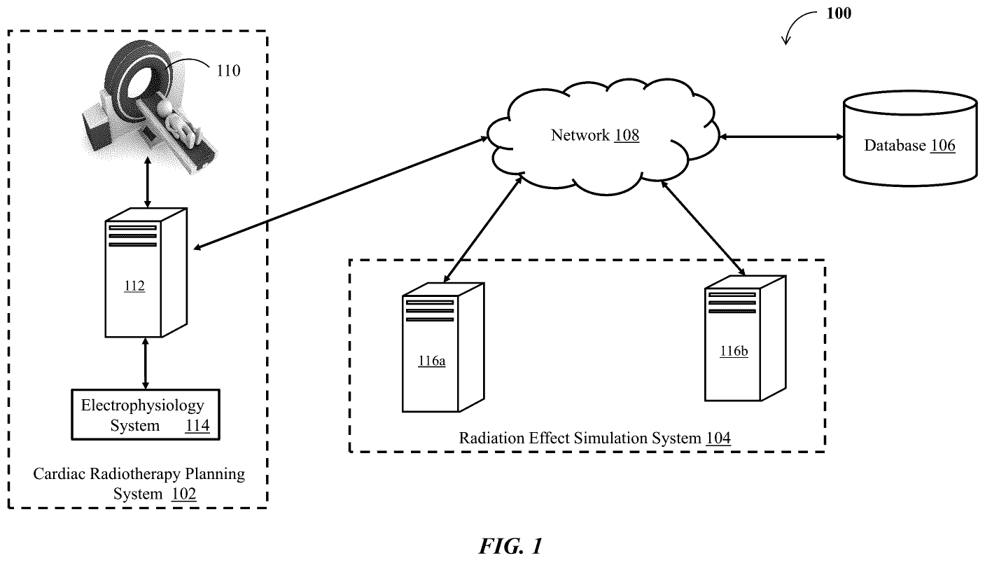

FIG. 1 illustrates an example computer environment 100 for planning of cardiac radiation therapy, according an example embodiment. In brief overview, the computer environment 100 can include a cardiac radiotherapy planning system 102, a radiation effect simulation system 104, a database 106 and a communication network 108. The cardiac radiotherapy planning system 102 can include an imaging device 110, an electrophysiology system 114 and one or more computing devices 112. The radiation effect simulation system 104 can include one or more computing devices such as computing devices 116a and 116b, referred to herein individually or collectively as computing device(s) 116. Computing device(s) 116 is/are configured to simulate the effect of radiation on a heart or the heart's electrical activities. The cardiac radiotherapy planning system 102, the radiation effect simulation system 104 and the database 106 can be communicatively coupled to each other through the communication network 108.

The communication network 108 may include a local area network (LAN), a wireless local area network (WLAN), a metropolitan area network (MAN), a wide area network (WAN), the Internet, a cellular network, a network of other type or a combination thereof. The network 108 may include both wired and wireless communications according to one or more standards and/or via one or more transport mediums. The communication over the network 108 may be performed in accordance with various communication protocols such as Transmission Control Protocol and Internet Protocol (TCP/IP), User Datagram Protocol (UDP), and IEEE communication protocols.

The cardiac radiotherapy planning system 102 can include one or more imaging devices 110, an electrophysiology system 114 and one or more computing devices 112. The imaging device 110 can include a computed tomography (CT) scanner, a magnetic resonance (MR) scanner, a positron emission tomography (PET) scanner or a combination thereof, among others. The imaging device 110 can acquire medical images of the heart of a patient over at least a portion of a cardiac cycle. In some implementations, the imaging device 110 can acquire a sequence of images depicting the heart motion or deformation over the cardiac cycle or a portion thereof. The acquired medical images can include CT images, computed tomography angiography (CTA) images, MR images, PET images, other types of medical images, or a combination thereof.

The electrophysiology system 114 is configured to perform electrophysiological studies of the patient, for example, to evaluate the heart's electrical system and to diagnose abnormal heart beats or arrhythmia. The electrophysiology system 114 can include one or more catheters, a plurality of wire electrodes and a computing device connected to the wire electrodes to record electrical signals. A doctor inserts the catheter in a vein in the groin of the patient, and then insert the wire electrodes into the patient's heart via the catheter and the vein. In some implementations, the electrophysiology system 114 can include a plurality of electrocardiogram (ECG) electrodes to be placed on the surface of the patient's body (e.g., chest) via a multi-electrode vest. Natural electric pulses of the heart can travel through the wire electrodes to be recorded by the computing device of the electrophysiology system 114. The computing device of the electrophysiology system 114 can send electrical signals through the electrodes to stimulate the heart tissue to try to cause the abnormal heart rhythm.

The computing device 112 can receive medical images from the imaging device 110 and electrophysiological study data from the electrophysiology system 114. The computing device 112 can also obtain other medical data of the patient, such as ECG data, blood pressure data and/or other patient data. The computing device 112 can be configured to run or execute radiation simulations as part of the radiotherapy planning. A radiotherapy planner can use the computer device 112 to simulate one or more sets of radiotherapy parameters to determine which set of parameters leads to a desired radiation dose distribution. The computer device 112 can also send acquired patient data, such as the medical images and the electrophysiology study data the database 106.

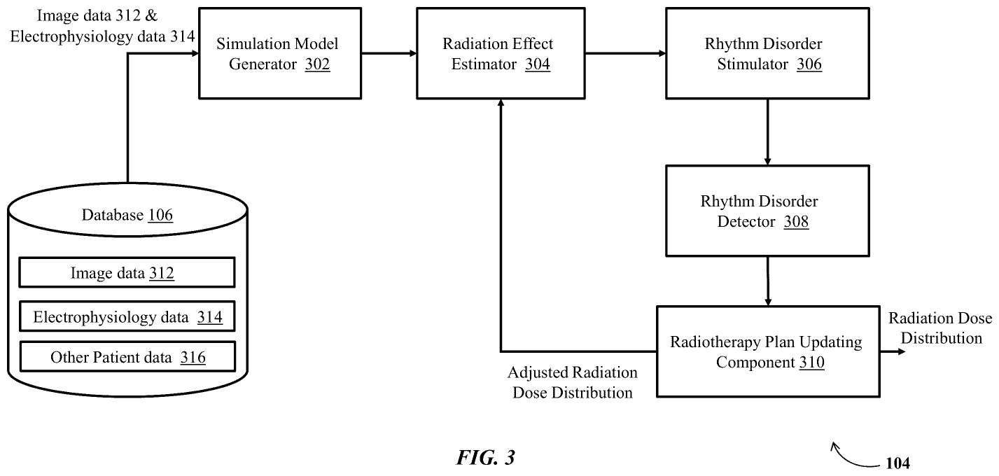

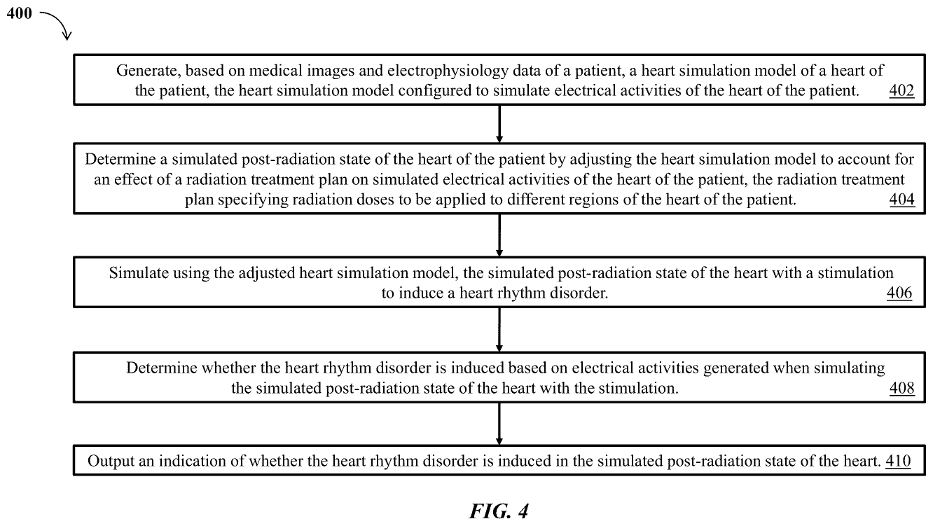

The radiation effect simulation system 104 (or respective computing device(s) 116) can be configured to use the acquired medical images and electrophysiology study data of the patient to generate a heart simulation model configured to mimic a patient's heart with respect to electrical activities. The effect of radiation on the patient's heart can be simulated as an adjustment in the parameters of the heart simulation model. The adjusted heart simulation model can be simulated with digital catheter stimulation to induce a heart rhythm disorder. If the heart rhythm disorder is successfully induced, then the radiation dose distribution may not be efficient in healing the heart. The testing for the potential recurrence of the heart rhythm disorder is a predictor of spontaneous recurrence of the heart rhythm disorder in the patient's heart post radiation treatment. The simulation of the effect of the radiation and the digital stimulation can help improve the radiation treatment plan and its efficacy. The functional properties of the radiation effect simulation system 104 (or respective computing device(s) 116) are discussed in further detail below in relation with FIGS. 3-4.

In some implementations, the computing device(s) 116 can be configured to execute computer instructions to perform any of the methods described herein or operations thereof. The computing device(s) 116 may generate and display an electronic platform to display information indicative of, or related to, the effect of radiation on the heart. The electronic platform may include a graphical user interface (GUI) for receiving input data and/or displaying indications of whether or not the heart rhythm disorder is successfully induced. An example of the electronic platform generated and hosted by the computing device(s) 116 may be a web-based application or a website configured to be displayed on different electronic devices, such as mobile devices, tablets, personal computer, and the like.

While FIG. 1 shows a network based implementation, it is to be noted that methods described herein can be implemented by a single computing device that receives the medical images and electrophysiological data of the patient and predicts the effect of radiation on the patient's heart according to methods described herein. The computer environment 100 is not necessarily confined to the components described herein and may include additional or alternative components, not shown for brevity, which are to be considered within the scope of the embodiments described herein. For instance, the computer environment 100 may include additional or alternative databases, for example within the cardiac radiotherapy planning system 102 or within the radiation effect simulation system 104. Also the number of computing devices within the cardiac radiotherapy planning system 102 or within the radiation effect simulation system 104 may vary according to various implementations.

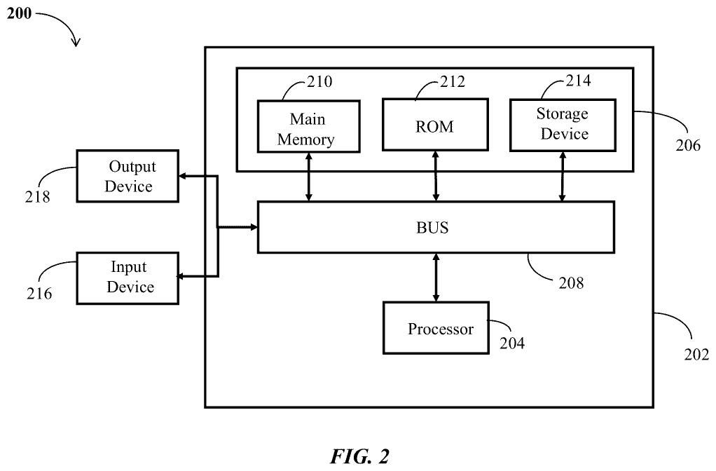

Referring to FIG. 2, a block diagram depicting one implementation of a system architecture for a computing system 200 that may be employed to implement methods described herein is shown, according to example embodiments. The computing system 200 can include a computing device 202. The computing device 202 can represent an example implementation of any of the devices 112 and/or 116 of FIG. 1. The computing device 202 can include, but is not limited to, a computed tomography (CT) scanner, a medical linear accelerator device, a desktop, a laptop, a hardware computer server, a workstation, a personal digital assistant, a mobile computing device, a smart phone, a tablet, or other type of computing device. The computing device 202 can include a one or more processors 204 to execute computer code instructions, a memory 206 and a bus 208 communicatively coupling the processor 204 and the memory 206.

The one or more processors 204 can include a microprocessor, a general purpose processor, a multi-core processor, a digital signal processor (DSP) or a field programmable gate array (FPGA), an application-specific integrated circuit (ASIC) or other type of processor. The one or more processors 204 can be communicatively coupled to the bus 208 for processing information. The memory 206 can include a main memory device 210, such as a random-access memory (RAM) other dynamic storage device, coupled to the bus 208 for storing information and instructions to be executed by the processor 204. The main memory device 210 can be used for storing temporary variables or other intermediate information during execution of instructions (e.g., related to methods described herein such as method 400) by the processor 204. The computing device 202 can include a read-only memory (ROM) 212 or other static storage device coupled to the bus 208 for storing static information and instructions for the processor 204. For instance, the ROM 212 can store medical images of patients, for example, received as input. The ROM 212 can store computer code instructions related to, or representing an implementation of, methods described herein. A storage device 214, such as a solid state device, magnetic disk or optical disk, can be coupled to the bus 208 for storing (or providing as input) information and/or instructions.

The computing device 202 can be communicatively coupled to, or can include, an input device 216 and/or an output device 218. The computing device 202 can be coupled via the bus 218 to the output device 218. The output device 218 can include a display device, such as a Liquid Crystal Display (LCD), Thin-Film-Transistor LCD (TFT), an Organic Light Emitting Diode (OLED) display, LED display, Electronic Paper display, Plasma Display Panel (PDP), or other display, etc., for displaying information to a user. The output device 218 can include a communication interface for communicating information to other external devices. An input device 216, such as a keyboard including alphanumeric and other keys, may be coupled to the bus 208 for communicating information and command selections to the processor 204. In another implementation, the input device 216 may be integrated within a display device, such as in a touch screen display. The input device 216 can include a cursor control, such as a mouse, a trackball, or cursor direction keys, for communicating direction information and command selections to the processor 204 and for controlling cursor movement on the display device.

According to various implementations, the methods described herein or respective operations can be implemented as an arrangement of computer code instructions that are executed by the processor(s) 204 of the computing system 200. The arrangement of computer code instructions can be read into main memory device 210 from another computer-readable medium, such as the ROM 212 or the storage device 214. Execution of the arrangement of computer code instructions stored in main memory device 210 can cause the computing system 200 to perform the methods described herein or operations thereof. In some implementations, one or more processors 204 in a multi-processor arrangement may be employed to execute the computer code instructions representing an implementation of methods or processes described herein. In some other implementations, hard-wired circuitry may be used in place of or in combination with software instructions to effect illustrative implementation of the methods described herein or operations thereof. In general, implementations are not limited to any specific combination of hardware circuitry and software. The functional operations described in this specification can be implemented in other types of digital electronic circuitry, in computer software, firmware, hardware or a combination thereof.

Referring now to FIG. 3, a block diagram of the radiation effect simulation system 104 shown in FIG. 1, according to an embodiment. The radiation effect simulation system 104 is a system for simulating the effect of radiation on a patient heart, including effects on the electrophysiological properties or electrical activities of the heart and/or pathological effects related to potential recurrence of a rhythm disorder associated with a pathological condition. In brief overview, the radiation effect simulation system 104 can include a simulation model generator 302 for generating a heart simulation model, a radiation effect estimator 304, a rhythm disorder simulator 306, a rhythm disorder detector 308 and a radiotherapy plan updating component 310.

The radiation effect simulation system 104 may include or may be connected to database 106. The database 106 can include image data 312, electrophysiology data 314 and/or other patient data 316. The image data 312 can include CT images, CTA images and/or other medical images of the patient. The electrophysiology data 314 can include electrophysiology study data of the patient depicting electrical activities of the patient's heart over at least a cardiac cycle or a portion thereof. For instance, the electrophysiology data 314 can include one or more electrical signals generated by the patient's heart and recorded by the electrophysiology system 114 via one or more electrodes. The electrophysiology system 114 can record the electrical signal(s) according to an invasive or non-invasive procedure. The other patient data 316 can include blood pressure data, ECG data, medical history data, demographic data or a combination thereof.

Each of the components 302, 304, 306, 308 and/or 310 can be implemented as a software component, hardware component, firmware component or a combination of software, firmware and/or hardware. For instance, any of these components can be implemented as computer code instructions that are executed by one or more processors, e.g., processor 204, to perform respective functional steps or processes. Any of the components 302, 304, 306, 308 and/or 310 can be implemented as a digital circuitry. The functional steps or processes associated with each of these components are described in further detail below in relation with FIG. 4.

FIG. 4 shows a flowchart illustrating an embodiment of a method 400 of simulating effect of radiation on electrical or electrophysiological properties of a heart for planning of cardiac radiation therapy, according to example embodiments. In brief overview, the method 400 can include generating a heart simulation model of a heart (step 402). For instance, one or more processors may generate, based on medical images and electrophysiology data of a patient, a heart simulation model of a heart of the patient, the heart simulation model configured to simulate electrical activities of the heart of the patient.

The method 400 may also include determining a simulated post-radiation state of the heart by adjusting the heart simulation model to account for an effect of a radiation treatment plan on simulated electrical activities of the heart (step 404). For instance, one or more processor may determine a simulated post-radiation state of the heart of the patient by adjusting the heart simulation model to account for an effect of a radiation treatment plan on simulated electrical activities of the heart of the patient, the radiation treatment plan specifying radiation doses to be applied to different regions of the heart of the patient.

The method 400 can include simulating the post-radiation state of the heart with a stimulation to induce a heart rhythm disorder (step 406). For instance one or more processors may simulate using the adjusted heart simulation model, the post-radiation state of the heart with a stimulation to induce a heart rhythm disorder.

The method 400 can include determining whether the heart rhythm disorder is induced (step 408). For instance, one or more processor may determine whether the heart rhythm disorder is induced based on electrical activities generated when simulating the post-radiation state of the heart with the stimulation.

The method 400 can include outputting an indication of whether the heart rhythm disorder is induced in the simulated post-radiation state of the heart (step 410). For instance, one or more processors may output an indication of whether the heart rhythm disorder is induced in the simulated post-radiation state of the heart.

The method 400 can be implemented by computing device(s) 116 or processor(s) thereof, such as processor 204.

Referring back to FIGS. 1-4, the method 400 can include the processor(s) 204 or the simulation model generator 302 generating a heart simulation model of a patient's heart using the image data 312 and the electrophysiology data 314 of a patient (step 402). Prior to the cardiac ablation procedure, the imaging device 110 can acquire image data 312 of the patient. The image data 312 can include cardiac gated CTA images, CT images, MR images, other types of medical images or a combination thereof. Also, the electrophysiology system 114 can record electrophysiological study data 314 of the patient depicting electrical activities of the patient's heart. The computing device 112 can store the image data 312 and the electrophysiology data 314 in database 106.

A doctor or other medical professional can mark or delineate the ROI to be radiated on one or more of the acquired medical images of the patient, for example, based on the image data 312 and the electrophysiology data 314. For instance, the doctor can manually identify (e.g., over a display of computing device 112) or mark the boundary of the ROI to be radiated. On some implementations, the computing device 112 can process the marked image(s) to refine the boundary of the ROI, for example, using image segmentation algorithms, object identification algorithms, other image processing algorithms or a combination thereof. The computing device 112 may use the refined (or originally marked) ROI boundary to identify the ROI in other non-marked images of the patient. In some implementations, the doctor can mark the ROI on all acquired medical images.

In some implementations, the ROI can include or can be one or more segments of a standardized N-segment model where N is an integer. For instance, the standardized N-segment model can include the standardized AHA 17-segment heart model. The standardized N-segment model represents a standard geometrical description or segmentation of the heart. The computing device 112 can identify the segments based on a parametric model of the heart chambers, for example, as described in “Four-Chamber Heart Modeling and Automatic Segmentation for 3-D Cardiac CT Volumes Using Marginal Space Learning and Steerable Features,” IEEE Transactions on Medical Imaging 27, no. 11 (November 2008) 1668-81, which is incorporated by reference herein. In some implementations, other standardized geometrical models of the heart can be used. An operator of the computing device 112 can identify the ROI as one or more segments of the standardized N-segment model, e.g., via a user interface displayed by the computing device 112.

The medical images acquired by the imaging device 110 can be two-dimensional images. The processor 204 or the simulation model generator 302 can generate a patient-specific geometrical model of the patient's heart using medical image data 312. The patient-specific geometrical model can include a three-dimension (3D) model depicting anatomical characteristics, e.g., shape, size and/or various anatomical regions, of the patient's heart. For instance, the processor 204 or the simulation model generator 302 can generate a 3D mesh of the patient's heart using the medical images of the patient. The processor 204 or the simulation model generator 302 can add one or more layers on top of the 3D mesh to reflect different regions or segments of the patient's heart.

In some implementations, the processor 204 or the simulation model generator 302 can register the electrophysiology data with the generated 3D model(s) of the patient's heart. In some implementations, the processor 204 or the simulation model generator 302 can combine the electrophysiology data with the generated 3D model(s) of the patient's heart as described in U.S. Pat. No. 9,463,072, the content of which is incorporated herein by reference. The electrical activity of the patient's heart varies spatially through the myocardium and over the surface of the heart. In general, the heart includes myocardial contractile cells and myocardial conducting cells. The myocardial conducting cells represent about one percent of the cells of the atria and ventricles, and they form the cardiac conduction system, which includes the sinoatrial (SA) node and the atrioventricular (AV) node. The myocardial conducting cells generate electrical impulses and propagate them throughout the heart. The myocardial contractile cells represent about 99 percent of the cells in the atria and ventricles. They also conduct electrical impulses, and are mainly responsible for contractions that pump blood through the body in response to the electrical impulses. As such, the heart acts as an electromechanical system where electrical impulses are generated by the cardiac conduction system and propagate into myocardial contractile cells to trigger contraction.

The processor 204 or the simulation model generator 302 can generate a simulation electrical model that is configured or structured to mimic or reproduce the electrical activities of the patient's heart. The simulation electrical model can be configured to generate signals similar to the signals of the electrophysiology data 314. The simulation electrical model can be a network of connected nodes where each node represents a block or a portion of the myocardium (or of the heart). Each node in the simulation electrical model can be associated with a respective activation signal and a respective conduction velocity. The respective activation signal can be a function that peaks or spikes relatively fast and then slowly decays over time to zero. Each activation signal of a corresponding node can be defined by a respective set of parameters.

In some implementations, each electrical activation signal can have multiple phases, and each phase can be defined by one or more corresponding parameters. In other words, each phase of an activation signal can be described in terms of a parameterized mathematical formulation. For instance, a first phase of an activation signal can be a steep ramp that is described or defined by a first parameterized mathematical formulation having one or more first parameters. A second phase of the activation signal can exhibit a slow decay, and can be described or defined by a second parameterized mathematical formulation having one or more second parameters.

The parameters of each activation signal or a phase thereof can be related to the underlying biology of the corresponding block or portion of the myocardium. For instance, the parameters of the activation signals can be set based on experimental data. Similarly, conduction velocity parameters corresponding to various nodes or various portions of the myocardium can be set based on experimental data. Results of experiments made on animals' hearts and/or humans' hearts can be used to set the parameters of the simulation electrical model, including the parameters of the activation signals and/or the conduction velocity parameters.

The conduction velocities associated with various nodes are correlated or dependent on the activation signals of the nodes or corresponding heart blocks or portions. Under healthy conditions, the steepness of the ramps of the activation signals are a major determinant of the conduction velocity. The steeper the ramp is the higher is the conduction velocity. In other words, how fast the electric signals propagate through the heart muscle or through the network of nodes depends on how steep are the ramps of the activation signals. As an illustrative example, if an activation signal associated with a corresponding node reaches its peak within 100 microseconds, and the threshold for activation of neighboring nodes is about half the peak amplitude, then the neighboring nodes will be activated in about 50 microseconds. However, if the activation signal reaches its peak in 500 microseconds, then neighboring nodes will be activated within about 250 microseconds. In the heart, a first cell gets activated or triggered (e.g., to exhibit the respective activation signal) by an activation signal of a neighboring cell. So the steeper the ramp of the activation signal of the neighboring cell, the faster the first cell gets activated, and that's how the properties of a single cell affects the conduction velocity.

Also, the decaying phase of an activation signal depends on the ramp or the peak of the same activation signal. The decaying phase can follow a predefined pattern that is described by a parameterized mathematical formulation, and the first value of the decaying phase depends on the peak of the activation signal. The higher the peak, the higher is the first value of the decaying phase of the activation signal. As such, the parameters of the mathematical formulation for the decaying phase change as the peak of the activation signal changes.

The processor 204 or the simulation model generator 302 can model the conduction velocity and the activation signals based on experimental data of healthy tissue and abnormal tissue. In particular, the parameterization functions can be predefined based on experimental data of healthy tissue and abnormal tissue. Also, the processor 204 or the simulation model generator 302 can set the conduction velocity parameters and the parameter values of the parameterization functions or respective phases using, for example, a lookup table or some other data structure maintaining parameter values for various tissue conditions (e.g., healthy tissue, tissue exhibiting VT, tissue exhibiting AFib, tissue exhibiting atrial flutter, tissue exhibiting atrial tachycardia, tissue exhibiting AVNRT, tissue exhibiting PSVT, tissue associated with Wolff-Parkinson-White syndrome or tumor tissue, among others). The processor 204 or the simulation model generator 302 can identify nodes corresponding to abnormal tissue based on the registration of the simulation electrical model with the 3D model of the patient's heart. Specifically, the processor 204 or the simulation model generator 302 can identify nodes associated with the ROI, and model their parameters accordingly. The processor 204 or the simulation model generator 302 can analyze the electrophysiology data 314 to extract measurements of electrical activity in the patient's heart. Measurements of electrical activity can include total activation time of the left or right ventricle, measurements extracted from ECG data such as QRS duration, electrical axis, QT interval duration, point-wise activation times and/or electrical voltage values as measured by catheter devices as part of electro anatomical mapping. The processor(s) 204 or the simulation model generator 302 can simulate the corresponding measurements of electrical activity and compares them with the measurements extracted from the electrophysiology data 314. The processor(s) 204 or the simulation model generator 302 can modify the conduction velocity and the activation signals to minimize the difference between the simulated and data-based measurements of the electrical activity. For example, an optimal value for the conduction velocity in one region of the heart can be estimated using an iterative algorithm, such as BOBYQA. In brief, multiple candidate values of the conduction velocity can be selected, and for each selection the corresponding simulated measurements of electrical activity can be produced. Based on which conduction velocity values produce simulated measurements of electrical activity closest to the data-based measurements, new candidate values can be generated. The algorithm can terminate when the difference between the simulated and data-based measurements of the electrical activity are below a certain threshold, or when a maximum number of candidates have been generated. Additional methods to modify the conduction velocity and the activation signals to minimize the difference between simulated and data-based measurements of the electrical activity are disclosed in U.S. Pat. Nos. 10,733,910, 10,483,005, 10,241,968, and 10,141,077, which are incorporated by reference herein.

Theoretically, nodes of the simulation electrical model can represent different cells of the heart. However, implementing the simulation electrical model at the cell level will result in a very complex model. Modeling the heart electrical system at coarse granularity (e.g., where each node corresponds to a separate myocardium block or portion) leads to a less complex model, while still providing accurate modeling. Usually neighboring healthy cells of the same type (e.g., myocardial contractile cells) have similar electrical properties.

The processor 204 or the simulation model generator 302 can register the simulation electrical model with the geometrical (or 3D) model. Each node in the simulation electrical model is mapped to its location in the 3D model. The heart simulation model includes the 3D model and the registered simulation electrical model. In some implementations, the processor(s) 204 or the simulation model generator 302 can use the other patient data 316 to generate the heart simulation model of a patient's heart. For example, the processor(s) 204 or the simulation model generator 302 can use the patient's age or patient's medical history to estimate one or more model parameters.

The method 400 can include the processor 204 or the radiation effect estimator 304 determining a simulated post-radiation state of the heart of the patient by adjusting the heart simulation model to account for an effect of a radiation treatment plan on simulated electrical activities of the heart of the patient (STEP 404). The radiation treatment plan can include a radiation dose delivery distribution specifying radiation doses to be applied to different regions of the heart of the patient. The radiation treatment plan may not be a complete plan at this stage. The radiation effect estimator 304 can receive the radiation treatment plan or the radiation dose delivery distribution from the cardiac radiotherapy planning system 102 or the computing device 112. In some implementations, the radiation dose delivery distribution can be the radiation dose prescribed by the patient's doctor. The radiation effect estimator 304 assumes that radiation has been delivered according to the radiation dose delivery distribution, and estimates the effect of the hypothetically delivered radiation on the electrical properties or electrical activities of the patient's heart.

Effects of radiation in the ROI or target volume can be simulated as changes in the electrophysiology properties of the cardiac tissue. For instance, radiation triggers upregulation in cardiac conduction proteins such as Nav1.5 and Cx43, which can be modeled as an increase in conduction velocity. A radiation dose-dependent increase in conduction velocity in the ROI can be defined based on experimental results, for example, as illustrated in FIG. 3 of Zhang, David M., Rachita Navara, Tiankai Yin, Jeffrey Szymanski, Uri Goldsztejn, Camryn Kenkel, Adam Lang, et al. “Cardiac Radiotherapy Induces Electrical Conduction Reprogramming in the Absence of Transmural Fibrosis,” Nature Communications 12, no. 1 (December 2021): 5558, which is incorporated by reference herein. This article is referred to hereinafter as “Zhang.” Also, the radiation effect estimator 304 can model new fibrosis potentially generated with high level of radiation as a conduction velocity reduction or even as zero conduction velocity.

Published experimental results, at least in Zhang, may indicate that radiation increases conduction velocity within surviving myocardium. Also, a relatively high level of radiation dose causes cells to die and become electrically non-active. In other words, if the radiation dose applied to heart tissue exceeds a given threshold, the tissue dies and becomes electrically inert (e.g., non-responsive to electrical simulation with no electrical capture nor propagation of electrical impulses). The radiation dose threshold can be determined from experimental results. In general, experimental results can include measured changes in conduction velocity for a plurality of delivered radiation doses.

The processor 204 or the radiation effect estimator 304 can interpolate the experimentally measured values. The processor 204 or the radiation effect estimator 304 can deduce or identify the radiation dose threshold that leads to fibrosis or dead tissue. The interpolated experimental results with the identified threshold can be maintained in one or more data structure(s), e.g., look up table(s) or linked list(s). The data structure(s) can maintain a mapping between radiation dose levels (or values) and corresponding changes in the conduction velocity (or corresponding conduction velocities). The data structure(s) can also maintain a mapping between radiation dose levels (or values) and corresponding changes in the activation signal parameters (or corresponding activation signal parameters). The mapping can include the corresponding changes in the parameters of each phase of the activation signal parameters (or the corresponding parameters of each phase of the activation signal). The variation in activation signal parameters (in terms of radiation dose levels) can be deduced from the variation in conduction velocity or can be defined based on experimental results measuring the change in activation signal parameters.

The processor 204 or the radiation effect estimator 304 can identify nodes of the simulation electrical model that belong to the ROI, e.g., based on the registration of the simulation electrical model with the 3D model, and determine based on the radiation dose distribution the radiation dose associated with the node or the corresponding block or portion of the heart. The processor 204 or the radiation effect estimator 304 can determine, for each node in the ROI, the change in conduction velocity and the change in activation signal parameters based on the corresponding radiation dose and the mapping(s) maintained in the one or more data structures. The processor 204 or the radiation effect estimator 304 can update, for each node in the ROI, the conduction velocity and the parameters of the activation signal. In the case where the activation signal is parameterized per phase, the processor 204 or the radiation effect estimator 304 can determine the change in parameters for each phase of the activation signal, and update the phase parameters accordingly.

Updating or adjusting the conduction velocity and the parameters of the activation signals leads to an updated or adjusted heart simulation model, which reflects the effect of the “delivered” radiation on the electrical properties (or electrical activities) of the heart. In other words, the updated or adjusted heart simulation model represents a simulated post-radiation state of the patient's heart as if radiated according to the radiation distribution (or the radiotherapy treatment plan). The initial conduction velocities and the initial activation signals represent the simulated pre-radiation state, while the adjusted conduction velocities and adjusted activation signals represent the simulated post-radiation state of the patient's heart.

The method 400 can include the processor 204 or the rhythm disorder simulator 306 simulating the simulated post-radiation state of the patient's heart with a stimulation to induce a heart rhythm disorder (step 406). The heart rhythm disorder can be associated with at least one of ventricular tachycardia (VT), atrial tachycardia, ventricular fibrillation, atrial fibrillation or other cardiac pathological condition. The processor 204 or the rhythm disorder simulator 306 can apply one or more digital catheter-based stimuli to the post-radiation state of the heart. For instance, in real life, a doctor would use a catheter to stimulate the heart with a fast train of stimuli to induce VT. If the radiation did not completely heal the heart, the heart will start contracting chaotically exhibiting an induced VT. An induced VT is a strong predictor of spontaneous VT post radiation.

The processor 204 or the rhythm disorder simulator 306 can digitally simulate the presence of a catheter that delivers very fast pacing to the heart simulation model. The digital simulation of the presence of the catheter can include applying a fast train of pulses to one or more nodes of the heart simulation model. The node(s) to which the fast train of pulses is applied can depend on the type of heart rhythm disorder to be induced. For instance, for VT, the stimulated node(s) corresponds to tissue(s) of the left or right ventricle. For atrial tachycardia, the stimulated node(s) corresponds to tissue(s) of one of the left or right atrium.

The method 400 can include the processor 204 or the rhythm disorder detector 308 determining whether the heart rhythm disorder is induced in the simulated post-radiation state of the heart (step 408). The processor 204 or the rhythm disorder detector 308 can determine whether the heart rhythm disorder is induced in the simulated post-radiation state of the heart based on electrical activities generated when simulating the simulated post-radiation state of the heart with the stimulation. The processor 204 or the rhythm disorder detector 308 can determine or calculate a simulated ECG signal of the heart simulation model, for example, as described in U.S. Pat. No. 9,463,072, which is incorporated by reference herein. The processor 204 or the rhythm disorder detector 308 can analyze the simulated ECG signal to determine whether the heart rhythm disorder is induced in the simulated post-radiation state of the heart.

In some implementations, the processor 204 or the rhythm disorder detector 308 can determine a frequency, a period or an autocorrelation signal of the simulated ECG signal. If the frequency, the period or the autocorrelation signal is not indicative of a normal periodic simulated ECG signal, the processor 204 or the rhythm disorder detector 308 can deduce that the heart rhythm disorder is induced in the simulated post-radiation state of the heart. If the heart rhythm disorder is induced that is a strong predictor that the radiotherapy treatment plan is not effective in healing the heart. In such case, either the radiotherapy treatment plan should be adjusted, or the patient should have an additional radiations session, e.g., with a new radiotherapy treatment plan, if the patient was already radiated according to the simulated radiotherapy treatment plan.

The method 400 can include the processor 204 or the radiotherapy plan updating component 308 outputting an indication of whether the heart rhythm disorder is induced in the simulated post-radiation state of the heart. In some implementations, the processor 204 or the radiotherapy plan updating component 308 can cause display of the simulated ECG signal on a display device, such as output device 218. A doctor would recognize based on the simulated ECG signal whether the heart rhythm disorder was induced. In some implementations, the processor 204 or the radiotherapy plan updating component 308 can display a text or a visual indicator indicative of whether the heart rhythm disorder was induced. The processor 204 or the radiotherapy plan updating component 308 can output an audio signal indicative of whether the heart rhythm disorder was induced.

In some implementations, the processor 204 or the radiotherapy plan updating component 308 can recommend or propose one or more modifications to the radiation treatment plan upon determining that the heart rhythm disorder is induced. The modifications can include modifying a radiation dose for tissue of the heart or the ROI specified in the radiation treatment plan or modifying (e.g., expanding) the ROI expected to receive radiation under the radiation treatment plan. For example, the processor 204 or the radiotherapy plan updating component 308 can examine the electrical activities of various nodes of the heart simulation model when stimulated to induce the heart rhythm disorder. The processor 204 or the radiotherapy plan updating component 308 can identify abnormal tissue in the ROI in the post-radiation state based on the electrical activities of corresponding nodes. If the electrical activity of some node is abnormal, the processor 204 or the radiotherapy plan updating component 308 can identify the node as abnormal and recommend increasing the radiation dose at the corresponding location or portion in the heart. The processor 204 or the radiotherapy plan updating component 308 can examine electrical activities at nodes corresponding to tissue surrounding the ROI. If the electrical activity of a node or corresponding tissue is determined to be abnormal or disorderly, the processor 204 or the radiotherapy plan updating component 308 can identify such tissue as abnormal or unhealthy and recommend expanding the ROI to include the surrounding tissue.

Once the radiation treatment plan (or radiation dose distribution) is updated, steps 404 to 410 can be repeated with the updated radiotherapy treatment plan (or radiation dose distribution). In some implementations, the processor 204 can execute steps 404 to 410 for a plurality of radiation treatment plans, and select the plan maximizing the chance of acute termination of the heart rhythm disorder (or the corresponding pathological condition) for applying to the patient. The radiation plan maximizing the chance of acute termination of VT is selected for execution in the patient. To estimate the chance of acute termination of the heart rhythm disorder (e.g., VT) one possible approach is to count the number of different programmed stimulation strategies resulting in sustained or un-sustained heart rhythm disorder in the simulated post-radiation state. The processor 204 can identify the radiation treatment plan leading to the smallest number of stimulations inducing the heart rhythm disorder as the plan maximizing the chance of acute termination of heart rhythm disorder (or the corresponding pathological condition).

In some implementations, the processor 204 or the radiotherapy plan updating component 308 may shrink the ROI (or reduce radiation doses) if the heart rhythm disorder is determined not to be induced. The processor 204 can execute steps 404 to 410 for the updated treatment plan, and determine whether the heart rhythm disorder is induced. The processor 204 may keep shrinking the ROI (or reducing radiation doses) and repeating steps 404 to 410 until the heart rhythm disorder starts to be induced. The processor 204 can select the treatment plan with the smallest ROI (or smallest radiation doses) that did not result in the heart rhythm disorder being induced. In some implementations, the radiation dose for at least some tissue in the ROI modifies the electrical properties of the tissue without destroying the tissue or rendering the tissue electrically inert.

In some implementations, method 400 can be used after radiation or during the radiation procedure. In such cases, if intra-interventional ECG measurements are available, the estimated conduction velocity at step 404 is further optimized after each radiation dose delivery to match ECG-based metrics. For instance, conduction velocity in each point of the heart can be scaled by a factor k which minimizes the discrepancy between simulated and measured ECG. In some implementations, a first dose delivery session is executed, followed by ECG measurement and corresponding update of the post-radiation state of the heart. The updated post-radiation state of the heart is used to optimize dose delivery in subsequent sessions.

One should note that the examples discussed in this specification are provided for illustrative purposes and are not to be interpreted as limiting. For example, other techniques can be used to estimate or adjust the effect of radiation on electrical properties of the heart. Also, other types of criteria can be used to determine whether the heart rhythm disorder is induced in the simulated post-radiation state of the heart.

Each method described in this disclosure, such as method 400 of FIG. 4, can be carried out by computer code instructions stored on computer-readable medium. The computer code instructions, when executed by one or more processors of a computing device, can cause the computing device to perform that method.

While the disclosure has been particularly shown and described with reference to specific embodiments, it should be understood by those skilled in the art that various changes in form and detail may be made therein without departing from the spirit and scope of the invention described in this disclosure.

While this disclosure contains many specific embodiment details, these should not be construed as limitations on the scope of any inventions or of what may be claimed, but rather as descriptions of features specific to particular embodiments of particular inventions. Certain features described in this specification in the context of separate embodiments can also be implemented in combination in a single embodiment. Conversely, various features described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated in a single software product or packaged into multiple software products.

References to “or” may be construed as inclusive so that any terms described using “or” may indicate any of a single, more than one, and all of the described terms.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain embodiments, multitasking and parallel processing may be advantageous.

What is claimed is:

1. A method of simulating radiation effect for cardiac ablation, the method comprising:
generating, by one or more processors, based on medical images and electrophysiology data of a patient, a heart simulation model of a heart of the patient, the heart simulation model configured to simulate electrical activities of the heart of the patient;
determining, by the one or more processors, a simulated post-radiation state of the heart of the patient by adjusting the heart simulation model to account for an effect of a radiation treatment plan on simulated electrical activities of the heart of the patient, the radiation treatment plan specifying radiation doses to be applied to different regions of the heart of the patient;
simulating, by the one or more processors, using the adjusted heart simulation model, the simulated post-radiation state of the heart with a stimulation to induce a heart rhythm disorder;
determining, by the one or more processors, whether the heart rhythm disorder is induced based on electrical activities generated when simulating the simulated post-radiation state of the heart with the stimulation; and
outputting, by the one or more processors, an indication of whether the heart rhythm disorder is induced in the simulated post-radiation state of the heart.

2. The method of claim 1, wherein the heart rhythm disorder is associated with at least one of ventricular tachycardia (VT), atrial tachycardia, ventricular fibrillation or atrial fibrillation.

3. The method of claim 1, wherein the electrical activities are defined in the heart simulation model using a plurality of parameters of a plurality of electrical activation signals, each electrical activation signal associated with a corresponding region of the heart of the patient, and wherein adjusting the heart simulation model includes:
adjusting parameters of electrical activation signals associated with a region of the heart of the patient expected to receive radiation under the radiation treatment plan.

4. The method of claim 3, wherein each electrical activation signal has multiple phases and each phase defined by one or more corresponding parameters, and wherein adjusting parameters of the electrical activation signal includes:
determining changes in parameters of each phase of the electrical activation signal due to an effect of the corresponding radiation dose on the region of the heart of the patient expected to receive radiation; and
modifying the parameters of each phase of the electrical activation signal according to the determined changes.

5. The method of claim 1, wherein the electrical activities are defined in the heart simulation model using one or more parameters of a conduction velocity.

6. The method of claim 5, wherein adjusting the heart simulation model includes adjusting at least one parameter of the one or more parameters of the conduction velocity.

7. The method of claim 1, wherein determining the simulated post-radiation state of the heart includes identifying tissue of the heart of the patient that is expected to become electrically inert in response to the radiation treatment plan.

8. The method of claim 1, wherein simulating the simulated post-radiation state of the heart with the stimulation to induce the heart rhythm disorder includes simulating a virtual catheter-based stimulation in the post-radiation state of the heart.

9. The method of claim 1 further comprising:
proposing one or more modifications to the radiation treatment plan upon determining that the heart rhythm disorder is induced.

10. The method of claim 9, wherein the one or more modifications to the radiation treatment plan include at least one of:
modifying a radiation dose for a first region of the heart specified in the radiation treatment plan; or
modifying a second region of the heart expected to receive radiation under the radiation treatment plan.

11. A system for simulating radiation effect for cardiac ablation, the system comprising:
one or more processors; and
a memory to store computer code instructions, the computer code instructions when executed cause the one or more processors to:
generate, based on medical images and electrophysiology data of a patient, a heart simulation model of a heart of the patient, the heart simulation model configured to simulate electrical activities of the heart of the patient;
determine a simulated post-radiation state of the heart by adjusting the heart simulation model to account for an effect of a radiation treatment plan on simulated electrical activities of the heart of the patient, the radiation treatment plan specifying radiation doses to be applied to different regions of the heart of the patient;
simulate, using the adjusted heart simulation model, the simulated post-radiation state of the heart with a stimulation to induce a heart rhythm disorder;
determine whether the heart rhythm disorder is induced in the simulated post-radiation state of the heart of the patient; and
output an indication of whether the heart rhythm disorder is induced in the simulated post-radiation state of the heart of the patient.

12. The system of claim 11, wherein the heart rhythm disorder is associated with at least one of ventricular tachycardia (VT), atrial tachycardia, ventricular fibrillation or atrial fibrillation.

13. The system of claim 11, wherein the electrical activities are defined in the heart simulation model using a plurality of parameters of a plurality of electrical activation signals, each electrical activation signal associated with a corresponding region of the heart of the patient, and wherein adjusting the heart simulation model includes:
adjusting parameters of electrical activation signals associated with a region of the heart of the patient expected to receive radiation under the radiation treatment plan

14. The system of claim 13, wherein each electrical activation signal has multiple phases and each phase defined by one or more corresponding parameters, and wherein in adjusting parameters of the electrical activation signal, the one or more processors are configured to:
determine changes in parameters of each phase of the electrical activation signal due to an effect of the corresponding radiation dose on the region of the heart of the patient expected to receive radiation; and
modify the parameters of each phase of the electrical activation signal according to the determined changes.

15. The system of claim 11, wherein the electrical activities are defined in the heart simulation model using one or more parameters of a conduction velocity.

16. The system of claim 15, wherein in adjusting the heart simulation model, the one or more processors are configured to adjust at least one parameter of the one or more parameters of the conduction velocity.

17. The system of claim 11, wherein when determining the simulated post-radiation state of the heart, the one or more processors are configured to identify tissue of the heart of the patient that is expected to become electrically inert responsive to the radiation treatment plan.

18. The system of claim 11, wherein when simulating the simulated post-radiation state of the heart with the stimulation to induce the heart rhythm disorder, the one or more processors are configured to simulate a virtual catheter-based stimulation in the post-radiation state of the heart of the patient.

19. The system of claim 11, wherein the one or more processors are further configured to:
propose one or more modifications to the radiation treatment plan upon determining that the heart rhythm disorder is induced.

20. The system of claim 19, wherein the one or more modifications to the radiation treatment plan include at least one of:
modifying a radiation dose for a first region of the heart specified in the radiation treatment plan; or
modifying a second region of the heart expected to receive radiation under the radiation treatment plan.
